(19) United States
(12) Patent Application Publication
(10) Pub. No.: US 2006/0167423 A1
(43) Pub. Date: Jul. 27, 2006

(54) Fluid collector

(76) Inventors: Marika Cherfas, Brooklyn, NY (US); Daniel Cherfas, Brooklyn, NY (US); Rut Cherfas, Brooklyn, NY (US); Henija Cherfas, Brooklyn, NY (US)

Correspondence Address: Marika Cherfas;Apt. 1B, 286 Corbin Place, Brooklyn, NY, 11235, US

(21) Appl. No.: 11/040,789

(22) Filed: Jan. 24, 2005

(51) Int. Cl. A61M 1/00

(52) U.S. Cl. 604/317

(57) ABSTRACT

A physiological fluid collector having an elastic body and an attachment mechanism. A first sensor is secured within an upper portion of the elastic body for detecting a predetermined fullness level of the elastic body. A second sensor is secured within a bottom portion of the elastic body for detecting a predetermined emptiness level of the elastic body. An emptying valve is located at the bottom of the elastic body and can be selectively opened and closed. When the first sensor indicates that the elastic body is full, the emptying valve is selectively opened and the content of the elastic body is emptied. When the second sensor indicates that the elastic body has been emptied, the emptying valve is closed and the collector is ready for the next use.

### BACKGROUND OF THE INVENTION

The invention relates to a collector device which can be placed internally into a wearer's body to collect physiological fluid.

Efforts have been made in the past to devise an internal fluid collector for collecting physiological fluid in people and animals. For example, tampons of various designs have been used to collect menstrual fluids in women. However, tampons do not separate the accumulated fluids from the internal organs of its wearer and, therefore, exposed these organs to harmful microorganisms accumulated in a tampon with the fluid. Thus, there has been a significant need in the art for a physiological fluid collector overcoming shortages of prior art collectors.

### SUMMARY OF THE INVENTION

In one of its aspects, the invention provides a physiological fluid collector having an elastic body and an attachment mechanism. A first sensor is secured within an upper portion of the elastic body for detecting a predetermined fullness level of the elastic body. A second sensor is secured within a bottom portion of the elastic body for detecting a predetermined emptiness level of the elastic body. An emptying valve is located at the bottom of the elastic body and can be selectively opened and closed. When the first sensor indicates that the elastic body is full, the emptying valve is selectively opened and the content of the elastic body is emptied. When the second sensor indicates that the elastic body has been emptied, the emptying valve is closed and the collector is ready for the next use.

In accordance with the present invention, the collector can be placed and attached internally in the desired area. As described below, the attachment is accomplished by evacuating the area between the collector and the internal bodily cavity thus securing the attachment mechanism to the body. The evacuation can be accomplished by using for example a syringe, a vacuum-extractor or a dental suction device. In the area of gynecology, the collector can be used to collect menstrual fluid by being positioned within woman's vagina around the opening of the cervix. The collector is delivered to the cervix using an ordinary gynecological mirror. The procedure can be done by the woman herself. It is also possible to connect the tubes of the collector to a decompressor can, which when activated would evacuate the area between the collector and the internal cavity.

As mentioned above, the collector is preferably equipped with two sensors. The first sensor detects the fullness of the collector and conveys this information to the wearer. The second sensor is activated during emptying of the collector and detects the zero level of the fluid within the collector below which the collector can self-detach itself from the cervix. The body of the collector is preferably made of rubber or another elastic material. Valves of the collector prevent fluid from leaking from the collector. The collectors can be made disposable single-use or multi-use. The fluid accumulates within the collector until the sensor indicates that the collector is full. If the collector is a single-use collector, the wearer can then dispose the collector and insert a new one, if necessary. A multi-use collector preferably includes a bottom valve allowing the wearer to empty the collector using a valve extender opening the bottom valve and extending to the outside of the body.

Depending on a woman's physiology, one collector can be sufficient for the entire duration of the menstrual period. The collector prevents the contact of the wearer's internal organs with the accumulated menstrual fluid which contains a plurality of harmful microorganisms. It also prevents any unpleasant smells from escaping from the collector.

The above aspects, advantages and features are of representative embodiments only. It should be understood that they are not to be considered limitations on the invention as defined by the claims. Additional features and advantages of the invention will become apparent in the following description, from the drawings, and from the claims.

### BRIEF DESCRIPTION OF THE DRAWINGS

The invention is illustrated by way of example and not limitation and the figures of the accompanying drawings in which like references denote like or corresponding parts, and in which:

FIG. 1 is a schematic diagram of the collector in accordance with the present invention.

FIG. 2 is a schematic diagram of the collector with an attached syringe.

FIG. 3 is a schematic diagram of a gynecological mirror used to insert the collector into a vagina.

FIG. 4 is a schematic diagram of the gynecological mirror with the collector being inserted into the vagina.

FIG. 5 is a schematic diagram of the collector inserted into the vagina.

FIG. 6 is a schematic diagram of the collector in the process of being attached to the cervix.

FIG. 7 is a schematic diagram of the collector collecting fluid into its elastic body.

FIG. 8 is a schematic diagram of emptying the collector.

FIG. 9 is a schematic diagram of the collector when the second sensor is activated.

FIG. 10 is a schematic diagram of manual removal of the collector

FIG. 11 is a schematic diagram of using a star spring as an attachment mechanism.

FIG. 12 is a schematic diagram of using a Bellville washer as an attachment mechanism.

FIG. 13 is a schematic diagram of using a extendible ring as an attachment mechanism.

FIG. 14 shows examples of specific dimensions of the extendible ring of FIG. 13.

FIG. 15 is a schematic diagram of using a tightening mechanism as an attachment mechanism.

FIG. 16 is a schematic diagram of using another tightening mechanism as an attachment mechanism.

### DETAILED DESCRIPTION OF THE PREFERRED EMBODIMENT AND THE DRAWINGS

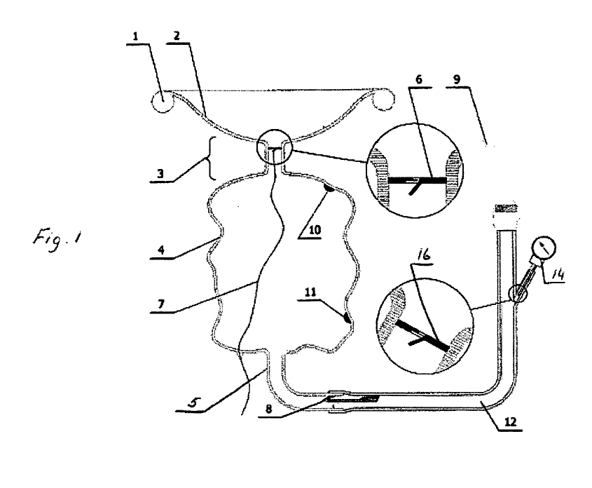

As shown in FIG. 1, the physiological collector of the present invention includes a body 4, preferably made of an elastic material, and a neck 3. An attachment mechanism 1 is connected to the vacuum cavity 2 ending at the top of neck 3. An entrance valve 6 is placed into the neck 3 for allowing the fluid to pass through it and into the interior of the elastic body 4. A string 7 extending to the outside and connected to the opening gate of the valve 6 can be provided for manual opening of the valve 6. Emptying canal 5 is connected to the bottom of the elastic body 4 and ends with an emptying valve 8.

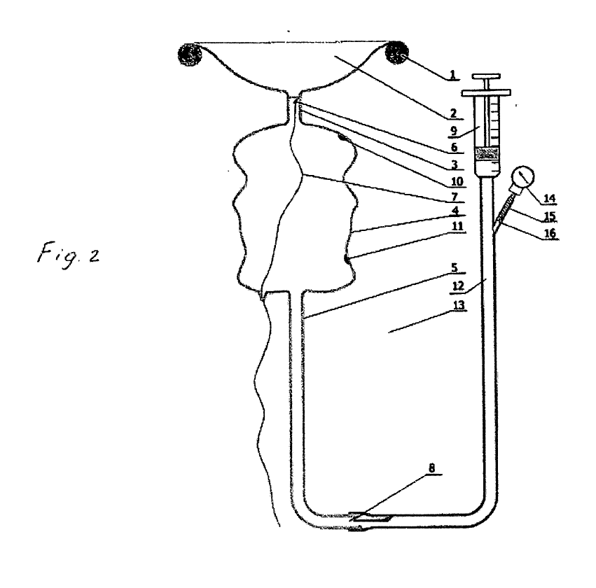

As shown in FIGS. 1 and 2, syringe 9 with its extender tube 12 may be selectively attached to the bottom opening of the emptying canal 5 such that the emptying valve 8 is controlled by the syringe 9. A vacuum-meter 14 having a shaft 15 and a valve 16 is connected to the extender tube 12 to observe and control the degree of decompression within the vacuum cavity 2.

At least two sensors are placed within the elastic body 4. The first sensor 10 preferably placed at the upper portion of the elastic body to detect fullness of the collector. The second sensor 11 is preferably secured at the bottom portion of the elastic body 4 to detect the emptying level of fluid within the collector. Any currently known sensor technology can be utilized with the present invention. For example, RFID chips can be utilized as sensors 10 and 11.

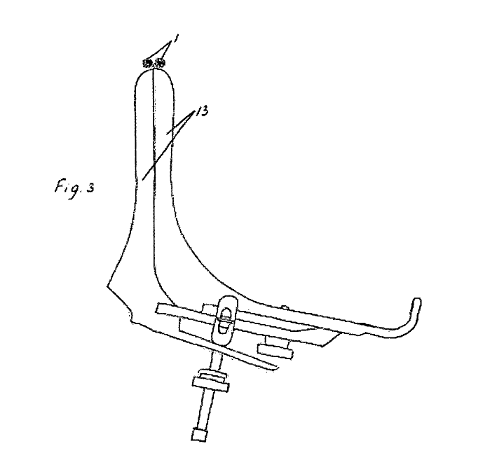

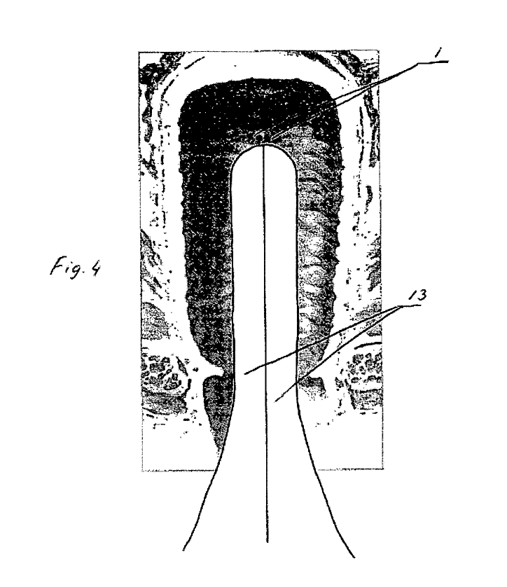

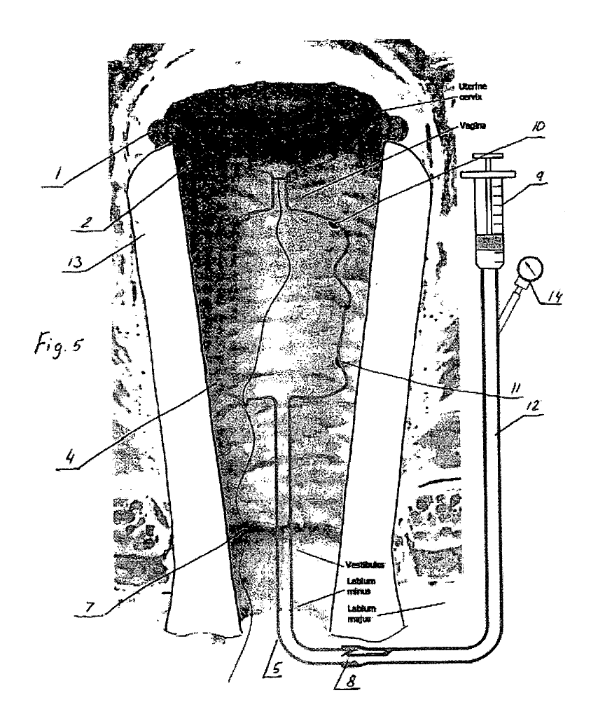

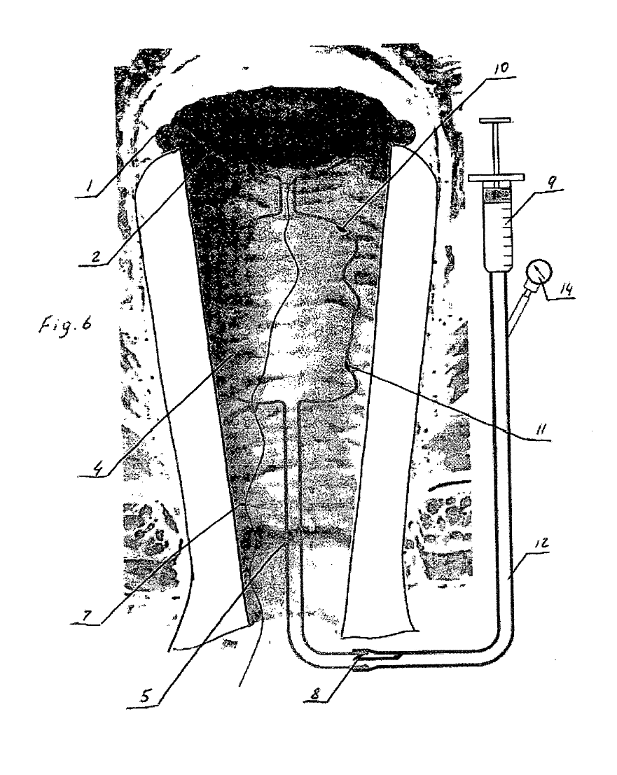

As shown in FIG. 3, to properly insert the collector into the vagina, the collector is preferably secured to a gynecological mirror such that the entire collector, except for the attachment mechanism 1, is located between the blades 13 of the mirror. The mirror can then be inserted into the vagina and moved to the opening of the cervix, as shown in FIG. 4. When the mirror is placed into the desired position, blades 13 are opened to release the attachment mechanism 1, which secures itself to the walls of the cervix, as shown in FIG. 5. To create vacuum between the cervix and the collector, i.e., within the vacuum cavity 2, the plunger of the syringe 9 is retracted from its barrel, as shown in FIG. 6. This process should be carefully controlled by observing the readings on the vacuum-meter 14. When the desired level of decompression is achieved, tube 12 of syringe 9 is disconnected from the bottom end of canal 5, thus closing the emptying valve 8 and preserving the vacuum between the collector and the cervix.

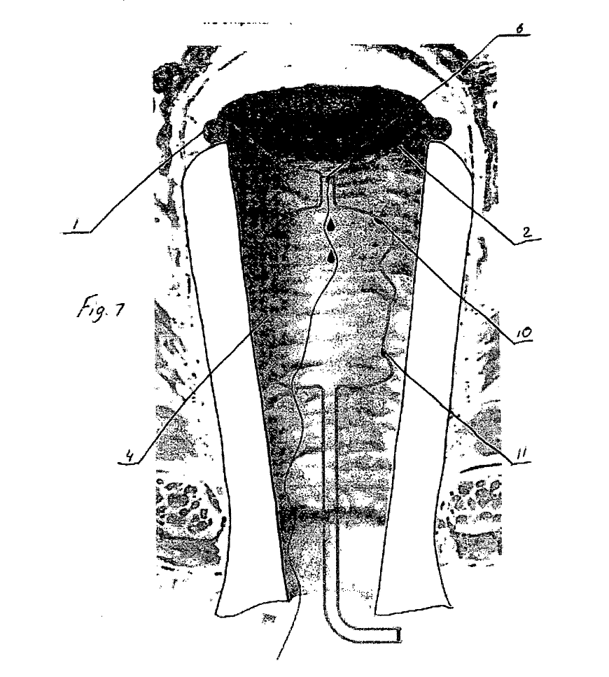

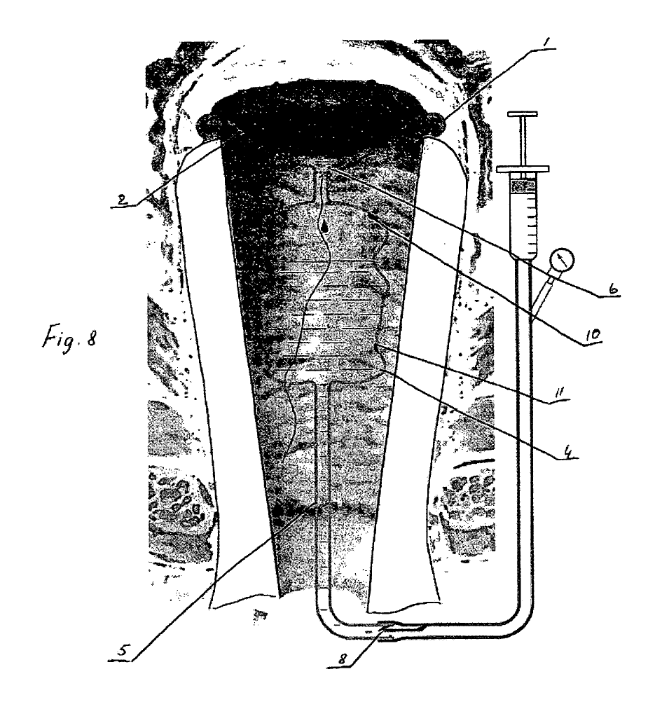

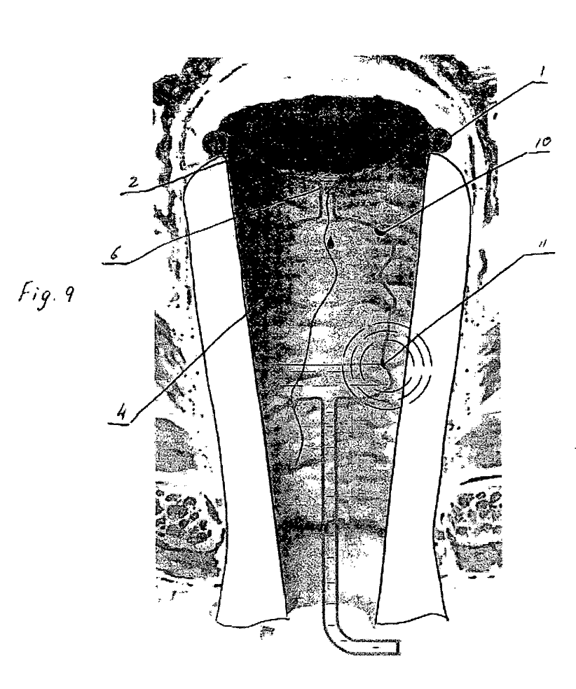

As shown in FIG. 7, when the volume of liquid within the vacuum cavity reaches a certain amount, valve 6 opens and allows the fluid to flow into and collect within the elastic body 4 of the collector. The fluid accumulation continues until the level of fluid reaches the first sensor 10. When activated by the fluid, first sensor 10 sends a signal to the wearer indicating that the collector is full. If the collector is of a multi-use variety, the wearer can connect a syringe or another appropriate vessel to the bottom opening of canal 5 such that the emptying valve 8 opens up and allows the fluid to flow away from the collector, as shown in FIG. 8. The discharge of the collected fluid continues until the fluid reaches the second sensor 11. As shown in FIG. 9, activated second sensor 11 sends a signal to the wearer that the fluid reached a predetermined emptiness level and that a critical level of air compression is reached. The emptying syringe is then promptly removed and emptying valve 8 is closed.

If the collector is of a single-use variety, the entire collector can be removed and disposed of when the signal of the first sensor is received by the wearer.

As shown in FIG. 10, the valve 6 can be opened manually using the string 7. Such manual opening will immediately remove the vacuum condition within the vacuum cavity and will allow the entire collector to be easily removed from the vagina. Such manual removal is especially beneficial when the physiological fluid collected in the collector needs to be examined.

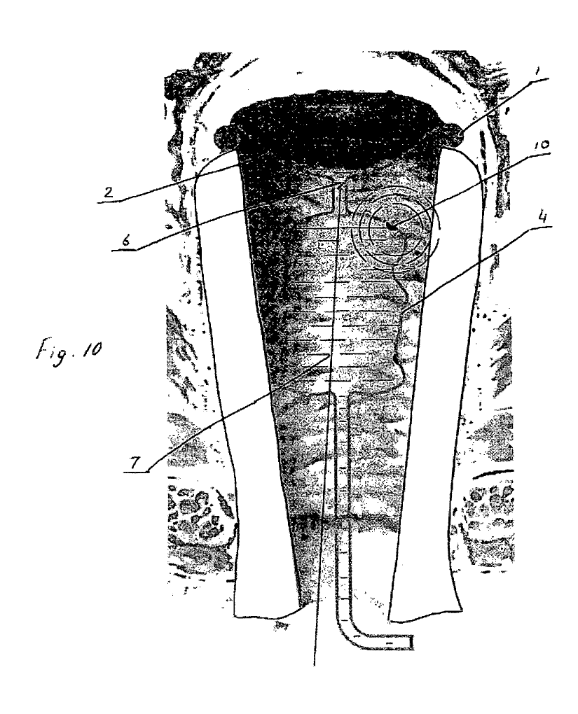

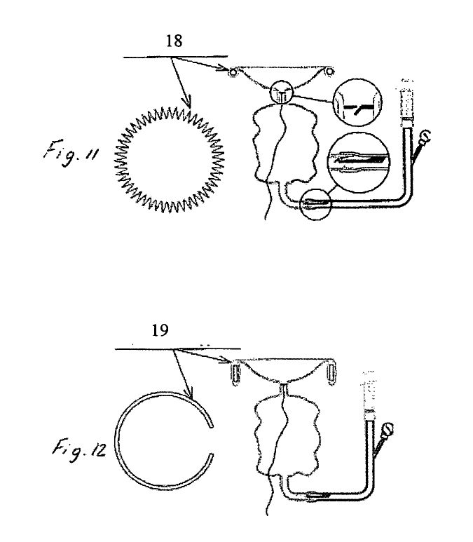

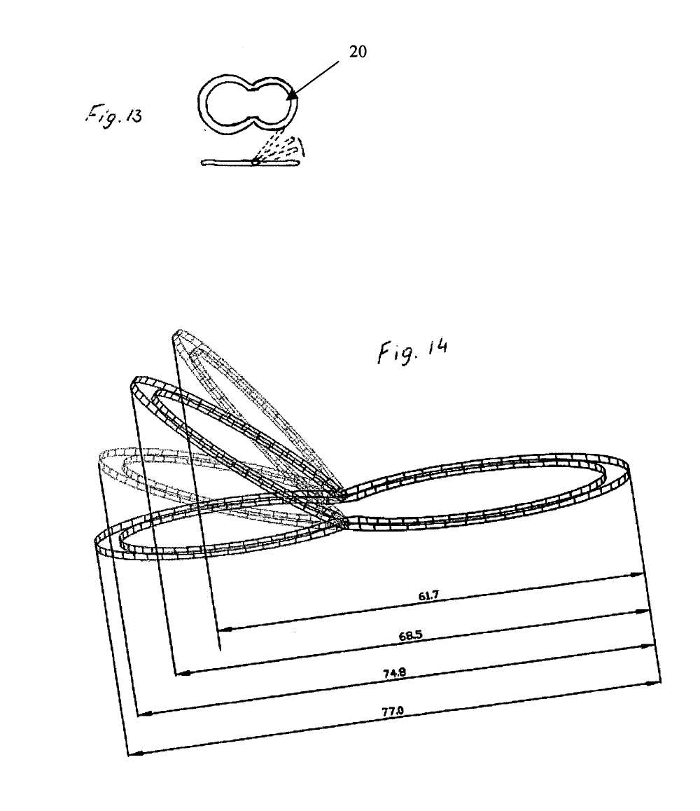

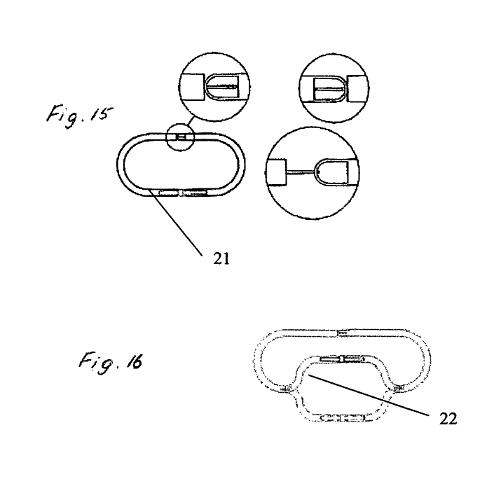

Several embodiments of the attachment mechanism 1 are shown in FIGS. 11-16. Specifically, the attachment mechanism can be implemented using a spring star 18 (FIG. 11), a Bellville washer 19 (FIG. 12), an extendible ring 20 (FIGS. 13-14), and a tightening mechanism 21 (FIGS. 15-16). Examples of different dimensions of the extendible ring (in millimeters) are shown in FIG. 14. High viscosity balms can also be used

For the convenience of the reader, the above description has focused on a representative sample of all possible embodiments, a sample that teaches the principles of the invention and conveys the best mode contemplated for carrying it out. It has also focused on a representative use of the invention. Other uses of the invention can be implemented by a person skilled in the art. For example, the described collector can be used in urology and proctology. The description has not attempted to exhaustively enumerate all possible variations. Other undescribed variations or modifications may be possible. For example, where multiple alternative embodiments are described, in many cases it will be possible to combine elements of different embodiments, or to combine elements of the embodiments described here with other modifications or variations that are not expressly described. Many of those undescribed variations, modifications and variations are within the literal scope of the following claims, and others are equivalent.

1. A physiological fluid collector comprising:
an elastic body further comprising an upper portion and a bottom portion;
an attachment mechanism;
a first sensor secured within the upper portion of the elastic body, said first sensor detecting a predetermined fullness level of the elastic body;
a second sensor secured within the bottom portion of the elastic body, said second sensor detecting a predetermined emptiness level of the elastic body; and
an emptying valve said emptying valve being configured to be selectively opened and closed,
wherein when the first sensor indicates that the elastic body is full, the emptying valve is selectively opened and the content of the elastic body is emptied, and wherein when the second sensor indicates that the elastic body has been emptied, the emptying valve is selectively closed.

2. The physiological fluid collector according to claim 1, wherein said attachment mechanism is a star spring.

3. The physiological fluid collector according to claim 1, wherein said attachment mechanism is a Bellville washer.

4. The physiological fluid collector according to claim 1, wherein said attachment mechanism is an extendible ring.

5. The physiological fluid collector according to claim 4, wherein said extendible ring can extend from 61.7 mm to 77 mm.

6. The physiological fluid collector according to claim 1, further comprising a vacuum cavity and a pressure valve located at a bottom of the vacuum cavity, said pressure valve being configured to be selectively opened and closed.

7. The physiological fluid collector according to claim 6 further comprising a manual opening mechanism connected to the pressure valve.
